(19) United States
(12) Patent Application Publication
(10) Pub. No.: US 2012/0046627 A1
(43) Pub. Date: Feb. 23, 2012

(54) NURSING PAD CLOTHING LINER

(76) Inventor: Rhonda L. AGNEW, Anton, TX (US)

(21) Appl. No.: 13/212,455

(22) Filed: Aug. 18, 2011

Related U.S. Application Data
(60) Provisional application No. 61/375,306, filed on Aug. 20, 2010.

(51) Int. Cl. A61F 13/15

(52) U.S. Cl. 604/358

(57) ABSTRACT

A nursing pad clothing liner for placing against a woman's breasts to protect from milk leakage.

### CROSS REFERENCE TO RELATED APPLICATIONS

This application claims priority to provisional application 61/375,306 filed on Aug. 20, 2010 by the present inventor and the application is hereby incorporated by reference in its entirety.

### STATEMENT REGARDING FEDERALLY SPONSORED RESEARCH OR DEVELOPMENT

Not Applicable

### NAMES OF PARTIES TO JOINT RESEARCH AGREEMENT

Not Applicable

### REFERENCE TO SEQUENCE LISTING

Not Applicable

### DESCRIPTION OF ATTACHED APPENDIX

Not Applicable

### BACKGROUND OF THE INVENTION

1. Field of the Invention

The disclosed invention relates to a nursing pad clothing liner, and more specifically to a nursing pad clothing liner that traverses across both breasts and can be worn without a brassiere.

2. Description of Related Art

Nursing pads or liners to absorb milk leakage are well known in the art for purposes of inserting one liner into each brassiere cup to absorb milk leakage from each breast. For example, U.S. Pat. No. 6,074,272 describes “a substantially flat oval shaped device to be inserted into the cup of a bra.” U.S. Pat. No. 6,039,629 entitled “Nursing Pad” and describes a multi-layer construction for a round pad for each breast. U.S. Pat. No. 5,017,174 similarly describes a two pad system for insertion into one's brassiere cups.

Although the above described pads are well known in the art, to date there has not been a practical solution for milk leakage at nighttime. Most women do not want to have to wear a brassiere while sleeping, however nearly all the known nursing pads are designed to be worn inside brassiere cups. U.S. Pat. No. 5,611,086 entitled “Nursing Garment” attempts to solve this problem by describing an actual vest type garment. This garment appears to be bulky and limits the user to a specific item of nighttime attire. Many women want to be able to sleep in a favorite nighttime tanktop, pajama top, t-shirt, or type of lingerie, (without wearing a bra) however the problem of milk leakage prevents them from doing so.

Another problem is that because the known nursing pads are primarily designed for use within a brassiere, they are consequently fairly thin and small in order to fit smoothly within the brassiere cup. Even when the pads are worn within the brassiere cups, they are rarely large or thick enough to absorb all nighttime leakage.

Yet another problem associated with the known two pad system (one pad to cover each breast) is that it allows no option for the user to wear the pads without also wearing a brassiere (whether while sleeping or awake). Many nursing women complain that they do not wish to wear a bra while at home, yet are forced to do so in order to control milk leakage, since all known pads require the use of a brassiere. Some known pads come with self adhesive such that they could potentially be applied to the inside of the user's clothing or to their skin, however this is not a viable option, for several reasons. First, the pads are small and if the user's clothing shifts at all they risk the pad moving off the areola region. Second, the known pads are too thin to be worn without a bra and may leak through. Third, without the concealing effect of a brassiere, the two pad system would be quite visible and odd looking through clothing, such as a shirt or tank top.

There is a need for an apparatus and method to enable nursing mothers to effectively control milk leakage without necessarily having to wear a brassiere or nursing brassiere, especially at night. Such a device and method needs to be comfortable, easily applied and removed, reasonably priced and available in a disposable form, although washable embodiments may be envisioned as well. A review of internet postings and blogs reveal that nursing women continually have two common complaints: 1) not being able to find pads that control the nighttime leakage sufficiently, and 2) having to wear a brassiere in order to utilize nursing pads. The disclosed nursing pad clothing liner solves both problems.

### NOTATION AND NOMENCLATURE

Certain terms are used throughout the following description to refer to particular method components. As one skilled in the art will appreciate, design and manufacturing companies may refer to a component by different names. This document does not intend to distinguish between components that differ in name but not function.

In the following discussion, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other intermediate devices and connections. Moreover, the term “method” means “one or more components” combined together. Thus, a method can comprise an “entire method” or “sub methods” within the method.

### SUMMARY OF THE INVENTION

The disadvantages shown in the prior art are solved by a nursing pad clothing liner that enables nursing mothers to wear their favorite attire while sleeping, without the need for a brassiere or nursing brassiere, while avoiding embarrassing and messy milk leakage.

It is an objective of the disclosed nursing pad clothing liner to protect a nursing mother's clothing or linens from unwanted milk leakage, without the mother having to don a brassiere.

It is an objective of the disclosed invention to provide a nursing woman protection from milk leakage with a nursing pad clothing liner that is comfortable and aesthetic in its appearance under clothing, even when a brassiere is not being worn.

It is an objective of the disclosed invention to provide a nursing woman protection from milk leakage with a nursing pad clothing liner that is effective and comfortable to wear while sleeping.

It is an objective of the disclosed invention to provide a nursing pad clothing liner that is easily applied to and removed from clothing or skin.

It is an objective of the disclosed invention to provide a nursing pad clothing liner that does not shift off the breast when outer clothing such as a shirt, shifts.

### BRIEF DESCRIPTION OF THE DRAWINGS

The drawings contained herein represent preferred embodiments of the invention and are not intended to limit the scope. For a detailed description of various embodiments, reference will now be made to the accompanying illustrative drawings in which:

FIG. 1 depicts a front view of the inner side of the disclosed nursing pad clothing liner.

FIG. 2 depicts the inner surface of an embodiment of the disclosed nursing pad clothing liner.

FIG. 3 depicts the outer surface of the disclosed nursing pad clothing liner.

FIG. 4 depicts the disclosed nursing pad clothing liner in place under a shirt.

FIG. 5A depicts an alternative embodiment of the disclosed nursing pad clothing liner, showing a back strap in the unsecured position.

FIG. 5B depicts an alternative embodiment of the disclosed nursing pad clothing liner, showing a back strap in the secured position.

### DETAILED DESCRIPTION OF THE PREFERRED EMBODIMENTS

The disclosed invention is a one-piece nursing pad or liner that covers both breasts such that a nursing woman can sleep without having to wear a brassiere in which individual pads are placed in each brassiere cup. In an alternate embodiment it may also be worn during waking hours by a woman who does not desire to wear a brassiere but still desires protection from milk leakage.

The present disclosure, because it is a one-piece liner traversing the entire area over and between the breasts, and because it is intended to not be worn with a brassiere, can thus be made thicker than the two part pads on the market, enabling it to absorb a larger quantity of milk leakage, which is especially beneficial while sleeping. It is also able to hold more milk leakage because of the fact that the one-piece liner is larger in coverage area than two individual pads, providing more absorbent area. Numerous benefits to this modification are apparent including the important aspect that a one-piece liner/pad covers more area than two small pads and will be more likely to catch all leakage as the user changes positions during the night. The one-piece liner is less likely to shift and move during the night. The one-piece liner allows the user to sleep comfortably without wearing a brassiere, while still accommodating milk leakage.

Even when worn during the day, without an accompanying brassiere, the one piece pad is far less likely to shift off the desired coverage area and is less obtrusively visible under clothing than the known two pad systems.

The disclosed nursing pad is intended to be disposable, although non-disposable embodiments utilizing washable fabrics may be contemplated as well.

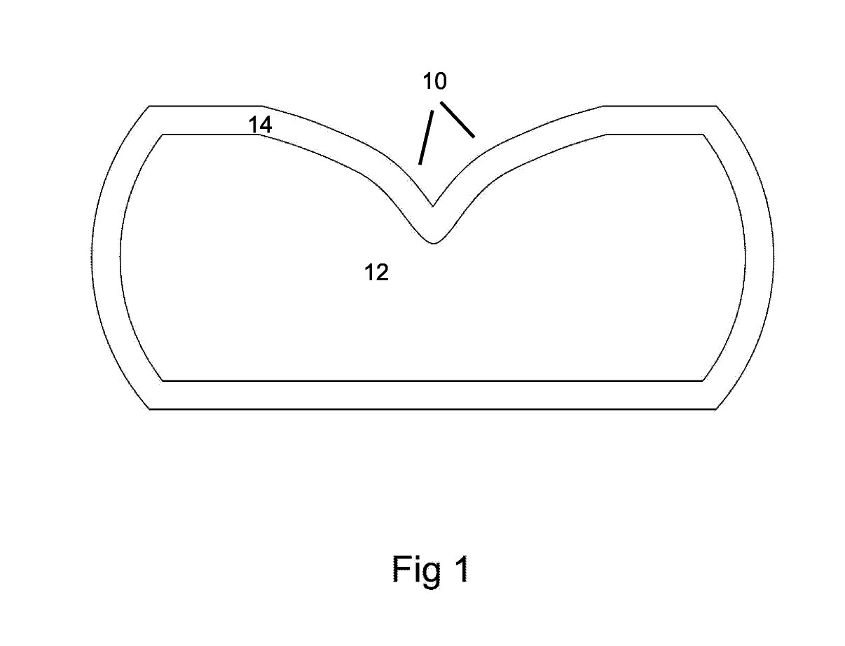

Turning to the figures, FIG. 1 depicts the disclosed nursing pad showing the inner surface of the pad 10 in a modified rectangular shape. The pad is comprised of at least one inner surface layer 12 capable of readily absorbing liquid (liquid permeable) and at least one outer surface layer 14 that holds liquid in (liquid resistant). In the preferred embodiment, the inner surface layer 12 that is closest to the skin is a liquid-permeable interfacing. The pad may comprise more than one layer including, but not limited to, two layers, or three layers, such as an inner layer that is capable of readily absorbing liquid, a middle layer that will absorb and hold liquid and an outer layer that is of a material conducive to holding the liquid in rather than letting it out. In an embodiment the middle layer is cotton fill sprinkled with sodium polycrylate and the outer layer surface is a liquid-resistant interfacing, although other suitable materials may be used in construction of the disclosed nursing pad.

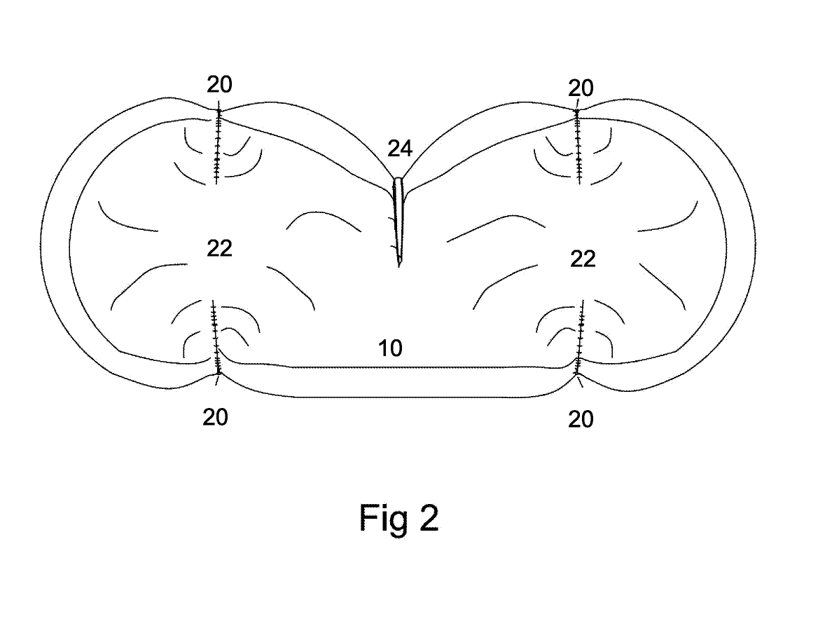

Any shape of the pad is acceptable provided it results in a one piece pad that traverses and fully covers the front region of both breasts, specifically covering at least the areola region. In addition to the modified rectangular shape disclosed, the pad may also be configured in a more contoured oval shape as reflected in FIG. 2, or other suitable shapes provided they meet the improvements described herein.

One of the difficulties that may be associated with a one-piece nursing pad clothing liner is that it could feel too bulky if it is not designed to conform to the shape of the breasts.

FIG. 2 reveals an alternate embodiment wherein the pad 10 is shown with four small vertical pleats 20 in the horizontal center of each side region 22 of the pad 10, two of the four pleats beginning at the top of the pad 10 and traveling a portion of the way down the side region 22 and an opposing two pleats beginning at the bottom of each side region 22 of the liner 10 and traveling a portion of the way up. There may also be a center pleat 24 starting at the top of the horizontal center of the liner 10 and traveling a partial way down the center of the liner 10. The number of pleats may vary in size and location on the liner provided there is at least one pleat with regard to this alternate embodiment.

The pleats enable the liner to contour to the shape of a woman's breasts, resulting in more of a concave surface to the interior of the liner and a convex surface exteriorly. This helps avert unwanted gapping and leakage, unwanted bulkiness and also prevents bothersome corners of the liner from sticking out.

In another alternate embodiment, the disclosed liner may have an elasticized trim along the perimeter of the liner such that the liner conforms to the woman's breast shape even more, for a still more comfortable and aesthetic fit. The liners may come in various sizes to accommodate the various sizes of women.

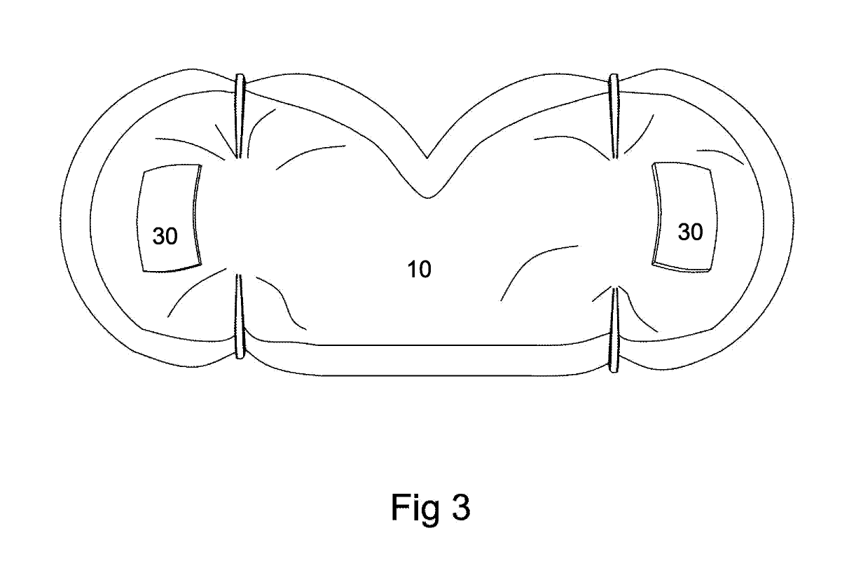

FIG. 3 illustrates the outer side of the liner 10 and further illustrates an adhesive patch 30 on either side of the liner 10 wherein the adhesive patch 30 can be used to adhere the liner 10 to an article of clothing. The liner 10 may also incorporate adhesive patches to the inner surface of the liner should the user wish to adhere the liner directly to her skin. The adhesive patches may also be in the form of a self adhesive strip that travels along the perimeter of the liner for adhering the liner to clothing or skin.

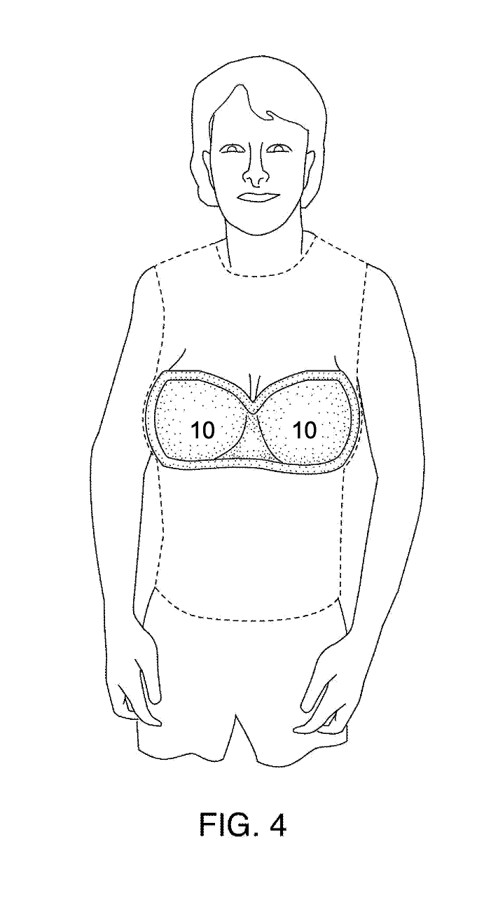

FIG. 4 illustrates the liner 10 in place on a woman, underneath a sleeveless shirt which is shown in phantom. The liner 10 is in place between the woman's breasts and the shirt. In the depicted embodiment the liner may be secured by any number of feasible ways including adhesive double sided tape securing the liner to the woman's skin or to the shirt itself. In the preferred embodiment, as a matter of practice, the user would remove her brassiere and adhere the outer side of the liner to the inner side of her article of clothing, for example a t-shirt, pajama top, lingerie top, shirt, tank top or other article of clothing. If desired the user may adhere the liner directly to her skin.

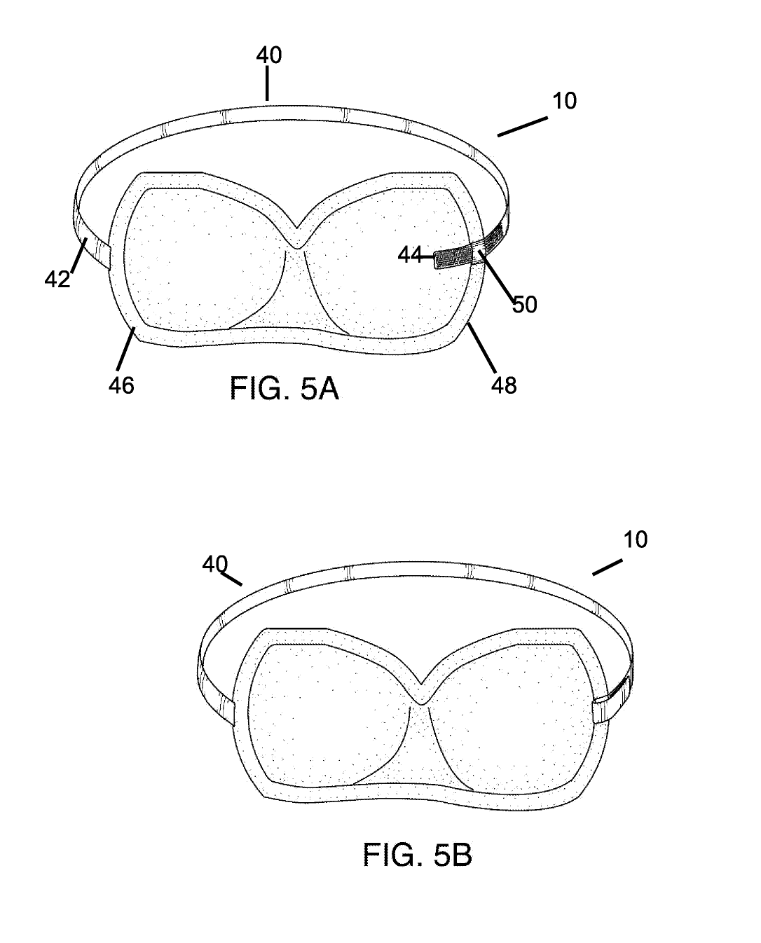

FIG. 5A depicts an alternative embodiment for securing the liner in place such that it covers the breasts. In FIG. 5A is shown a strap 40 having a first end 42 and a second end 44, wherein the first end 42 is fixedly attached at one side edge 46 of the liner 10 and the second end 44 is removable secured to the opposing side edge 48 of the liner 10 by first traveling through an aperture 50 and then securing back on itself by means of a hook and loops type of fastening, such as Velcro™ means or other feasible fastening means. The strap itself may be elasticized.

FIG. 5B depicts the embodiment described in FIG. 5A however it shows the strap secured in place as described above. Other means of holding the liner in place may be utilized provided they are in keeping with the objectives described herein.

Once the liner is securely in place, the user may then sleep or lounge comfortably without fear of milk leakage onto her clothing or bedding, and without the need for wearing a brassiere. As needed the user may change the disposable or non-disposable liner when it becomes saturated with milk.

The disclosed nursing pad clothing liner solves the problems currently encountered with known nursing liners by offering a liner that, due to its novel features, may be used without a brassiere. The liner is comfortable and effective. The liner may be made as thickly as desired since the liner is not constrained by a need to fit it into a brassiere. The thickness may be adjusted by adhering multiple liners together or manufacturing the liners in various thicknesses, similar to the way sanitary napkins are offered in various thicknesses.

While the disclosed method and apparatus has been described in conjunction with the preferred embodiments thereof, many changes, modifications, alterations and variations will be apparent to those skilled in the art. The invention should therefore not be limited to the particular preferred embodiment disclosed but should include all embodiments that could fall within the scope of the claims.

Accordingly, the preferred embodiments of the invention shown in the drawings and described in detail above are intended to be illustrative, not limiting, and various changes may be made without departing from the spirit and scope of the invention as defined by the claims set forth below.

What is claimed is:

1. A nursing pad clothing liner for placing against a woman's breasts to protect from milk leakage on clothing, said liner comprising:
at least one inner layer of liquid permeable material for absorbing and holding milk leakage, the at least one inner layer being of at least sufficient length and width to cover the front of both breasts;
at least one outer layer of liquid resistant material for preventing the milk leakage from migrating outside the liner, the at least one outer layer being of at least the same length and width than the at least one inner layer;
wherein said liner is generally rectangular in shape;
wherein said liner has a means for removably securing the liner in place against the breasts;

2. The liner of claim 1 wherein said liner further comprises at least one vertical pleat for conforming the liner to the shape of the breasts.

3. The liner of claim 1 wherein said liner further comprises at least two vertical pleats for conforming the liner to the shape of the breasts.

4. The liner of claim 1 wherein the means for removably securing the liner consists of adhesive tape.

5. The liner of claim 1 wherein the means for removably securing the liner consists of at least one strap adhered to the liner, the at least one strap traveling around the woman's back to hold the liner against the front of the breasts.

6. The liner of claim 1 further having an elasticized perimeter.

7. A nursing liner for capturing milk leakage while a nursing woman sleeps, said nursing liner comprising:
at least one inner layer of liquid permeable material for absorbing and holding milk leakage, the at least one inner layer being of at least sufficient length and width to cover the front of both breasts;
at least one outer layer of liquid resistant material for preventing the milk leakage from migrating outside the liner, the at least one outer layer being of at least the same length and width than the at least one inner layer;
wherein said liner is generally rectangular in shape;
wherein said liner further comprises at least one vertical pleat for conforming the liner to the shape of the breasts.

8. The liner of claim 7 wherein said liner further comprises at least two vertical pleats for conforming the liner to the shape of the breasts.

9. The liner of claim 7 wherein said liner further comprises a strap that encircles the back for securing the liner in place against the breasts.

10. The liner of claim 7 wherein the means for removably securing the liner consists of adhesive tape.

11. The liner of claim 7 further having an elasticized perimeter.

12. A method for preventing milk leakage from soiling a woman's clothing, the method comprising:
covering the front of both breasts with a nursing pad clothing liner that is generally rectangular in shape and comprises at least one inner layer of liquid permeable material for absorbing and holding milk leakage, the at least one inner layer being of at least sufficient length and width to cover the front of both breasts; and at least one outer layer of liquid resistant material for preventing milk leakage from migrating outside the liner, the at least one outer layer being of at least the same length and width than the at least one inner layer, and;
removably securing the liner against the breasts.

13. The method of claim 12 wherein said liner further comprises at least one vertical pleat for conforming the liner to the shape of the breasts.

14. The method of claim 12 wherein said liner further comprises at least two vertical pleats for conforming the liner to the shape of the breasts.

15. The method of claim 12 wherein the means for removably securing the liner consists of adhesive tape.

16. The method of claim 12 wherein the means for removably securing the liner consists of at least one strap adhered to the liner, the at least one strap traveling around the woman's back to hold the liner against the front of the breasts.

17. The method of claim 12 further having an elasticized perimeter.
